(19) United States
(12) Patent Application Publication
(10) Pub. No.: US 2003/0152523 A1
(43) Pub. Date: Aug. 14, 2003

(54) Pharmaceutical composition for pulmonary delivery

(76) Inventors: Gary Peter Martin, East Sussex (GB); Xian Ming Zeng, Mitcham Surrey (GB)

Correspondence Address: Matthew P Vincent, Ropes & Gray, One International Place, Boston, MA, 02110-2624, US

(21) Appl. No.: 10/203,266

(22) PCT Filed: Feb. 7, 2001
(86) PCT No.: PCT/GB01/00489

(30) Foreign Application Priority Data
Feb. 8, 2000 (GB) 0003935.4

(51) Int. Cl. A61K 31/573; A61L 9/04; A61K 9/14

(52) U.S. Cl. 424/46; 514/179

(57) ABSTRACT

A dry particulate pharmaceutical composition for pulmonary administration is prepared by spray-drying a hydrophobic active agent (e.g. declomethasone dipropionate) with a small quantity of pharmaceutically acceptable hydrophilic polymer (e.g. poly (vinyl) alcohol). This mitigates aggregation of the particles of active agent, which otherwise limits the desirable find particles dose available.

The present invention relates to pharmaceutical compositions for pulmonary delivery in the form of spray-dried particles, and methods of preparation thereof.

### BACKGROUND

Inhaled corticosteroids are recommended to be initiated at an early stage in the treatment of all asthmatic patients^{(1,2)} with higher initial dosages being subsequently tapered to the lowest effective dosage.⁽³⁾ However, there are concerns regarding the safety of corticosteroids inhaled at high dose⁽⁴⁾ since drugs such as beclomethasone dipropionate (BDP) administered in this manner have been found to induce systemic side effects such as adrenocortical suppression, skin changes (thinning, bruising) and cataract formation. Ideally, therefore all of the administered dose of corticosteroid should be delivered to the site of action in the respiratory tract so as to obtain the localized therapeutic effects whilst minimizing the amount gaining access to the systemic circulation. The pressurized metered dose inhaler (pMDI) is the most widely used device for administering corticosteroids to the respiratory tract but the chlorofluorocarbon (CFC)-containing pMDIs are gradually being phased out in order to comply with the United Nations Environmental Programme.⁽⁵⁾ Thus, there has been a resurgence of interest in employing dry powder inhalers (DPIs) in pulmonary drug delivery but DPIs are notorious for the relatively low delivery efficiency of drugs to the lung. For example, the Rotahaler, Spinhaler and Diskhaler were reported to deliver only about 10% of the total administered dose to the lower airways.⁽⁶⁾ The majority of the drug is deposited in the upper airways and most of this is eventually swallowed and absorbed systemically via the gastrointestinal tract.⁽⁷⁾ Thus, the optimization of drug delivery from DPIs to the lower airways will not only increase the therapeutic effects but also reduce any possible side effects. This is particularly of clinical significance for BDP, which was found to produce very low respirable fractions after delivery from DPIs.⁽⁶⁾ Most dry powder formulations for inhalation are comprised of fine drug blended with a coarser carrier. α-Lactose monohydrate has been employed most frequently as the carrier and it is usually fractionated so as to have a specific size range such as 63-90 μm for this purpose.⁽⁵⁾ On inhalation, the drug particles must be dissociated from the carrier and dispersed into the air stream, which then carries the air-borne particles into the lung. The drug particles are usually present in low concentrations, with a drug to carrier ratio of 1:67.5, w/w, being typical.⁽⁸⁾ Any change in the physico-chemical properties of the drug or carrier particles has the potential to alter the drug deposition profile.

### THE INVENTION

The present invention provides a particulate pharmaceutical composition for pulmonary delivery, which comprises a hydrophobic pharmaceutically active agent which has been spray-dried with a small quantity of a pharmaceutically acceptable hydrophilic polymer to form spray-dried particles.

The invention also provides a corresponding method for preparing the spray-dried particles.

Thus, it was the aim of the current study to attempt to reduce the hydrophobic interaction that exists between BDP particles. Such interaction tends to induce aggregate formation and this potentially limits the fine particle dose available for pulmonary delivery. BDP was also selected for study since it provides a representative hydrophobic drug for which there is a requirement for delivery via the pulmonary route. The objectives were to adsorb hydrophilic polymer to individual drug particles and to prepare a model dry powder formulation containing modified drug and lactose. Finally it was the aim to determine the deposition of drug from the formulation in a twin stage impinger. The polymer selected for study was poly(vinyl) alcohol (PVA), a polymer acceptable as an excipient for use in formulated pMDIs. It was intended to apply the polymer to the particles suspended in an aqueous solution by spray drying.

The pharmaceutically acceptable hydrophilic polymer is any such polymer known as being suitable for pulmonary administration. As mentioned, poly(vinyl)alcohol is preferred. Other suitable hydrophilic polymers include carbomers (such as Carbopol 934), glycosaminoglycans (such as hyaluronic acid), dextrans, alginates, hydrophilic cellulosic-based polymers (such as sodium carboxymethylcellulose) and polyethylene glycols. Generally, the ratio of active agent to hydrophilic polymer is in the range 1:0.05 (i.e. about 5%) to 1:0.1 (i.e. about 10%) by weight. Ratios up to 1:0.2 have been successfully spray-dried.

The size of the spray-dried particles will be chosen for optimal pulmonary administration. As is well known, the aerodynamic particle size for administration to the lung is in the range 1 to 6 μm as determined by impactor techniques (and less than 20 μm for nasal administration). According to the British Pharmacopeia (BP) specification the fine particle dose (FPD) is less than 6.4 μm (see Tables 1-3).

The active agent may be any agent suitable for pulmonary delivery. Suitable active agents for delivery to the lung to achieve a local effect include beta 2 agonists (e.g. salmeterol, salbutamol), corticosteroids (e.g. declomethasone dipropionate, budesonide, fluticosone dipropionate), anticholinergic drugs (e.g. ipratropium bromide) and leukotrienes.

Active agents may also be administered to the lung in order to achieve systemic medication of the patient. Suitable active agents include peptides (e.g. insulin, calcitonin), antisense therapeutics and genes for gene therapy delivery.

The pharmaceutical composition may also include a pharmaceutically acceptable carrier. Lactose is a preferred carrier. Other carriers include mannitol, arabinose, xylitol and dextrose, or monohydrates thereof; maltose, sucrose, dextrin and dextran. The carrier preferably has a specific size range of 63-90 μm. In a preferred formulation, the ratio of spray-dried particles to carrier is 1:50 to 1:85 w/w (e.g. substantially 1:67.5). The hydrophilic polymer is usually 0.073 to 0.146% of the total composition (including carrier).

The spray-dried particles may be produced using known techniques. In particular, the particles of hydrophobic active agent (which is generally water insoluble) are usually dispersed in an aqueous solution of the hydrophilic polymer. The dispersion is then spray-dried in known manner.

The invention will now be described by way of example only with reference to the following Examples.

### DETAILED DESCRIPTION OF PREFERRED EMBODIMENTS

It was the aim of the current study to attempt to reduce the hydrophobic interaction that exists between BDP particles. Such interaction induces aggregate formation and potentially limits the fine particle dose available for pulmonary delivery. The objectives were to adsorb hydrophilic polymer to individual drug particles and to prepare a model dry powder formulation containing modified drug and lactose. A suspension of 1% w/v BDP in water containing either 0.05% or 0.1% PVA ((MWt 9-10 kDA) was spray dried to produce particles (volume mean diameter (VMD) 4.2 μm as determined by an Aerosizer time of flight particle sizing apparatus obtained from Amherst Process Instruments, Hadley, Mass., USA) suitable for intended delivery to the lung. The micronised drug (VMD 4.1 μg) or spray dried drug was blended with lactose in a ratio 1:67.5 parts by weight and the resultant formulations filled into gelatin capsules for arosolisation via a Rotahaler device to a twin stage impinger (TSI) operated at 60 L min⁻¹. The Fine Particle Dose (FPD) (as determined from by the amount of drug reaching the lower stage of the TSI) was increased from 20.2 μg when micronised drug was employed to 46.7 μg when the lower PVA concentration was employed. The FPD was increased further, to 72.9 μg, when the formulation containing the drug spray-dried with the higher concentration of PVA was aerosolised.

The results obtained in this study suggest that the approach of spray-drying a hydrophobic drug with small quantities of a pharmaceutically acceptable hydrophilic polymer holds great promise in the formulation of other, similar materials as dry powders intended for pulmonary delivery.

### METHODS

1. Preparation of coarse Lactose:

Lactose crystals (Batch no. S648090, Borculo Whey Ltd, Chester, UK) were sieved using an air-jet sieve (Alpine, Ausberg, Germany). Lactose crystals (approximately 50 g) were first passed through a test sieve with an aperture width of 90 μm (Endecotts Ltd, London, UK) for 15 min and the sieved powder was then passed through a 63 μm sieve for a further 15 min. The powder retained on the 63 μm sieve was subjected to the same procedure in order to ensure that the majority of particles fell within the size range 63-90 μm. The sieved powder was stored in a sealed jar over silica gel until required for further use later. 2. Preparation of Spray Dried Beclomethasone Diproprionate (BDP):

2.1) Preparation of Suspensions for Spray Drying:

Poly(vinyl) alcohol (PVA), 80% hydrolysed, average molecular weight 9,000-10,0000 was supplied by Aldrich Chemical Company. PVA (0.05 or 0.1 g) was dissolved in 100 ml of distilled water at 50° C. 1 g BDP was dispersed in each PVA solution. Stirring and heating at 50° C. was continued for 20 min to obtain an homogeneous suspension of BDP.

2.2) Spray-drying of Suspensions of PVA and BDP

Each of the suspensions was spray dried using the Niro Atomiser spray-drier (Coppenhagen, Denmark No.1339). The spray-drier was run under the following conditions: Speed : 38,000 rpm, Feed rate : 800 ml h⁻¹, Heat setting: level 4 (inlet temperature 180° C., outlet temperature 90° C.).

The spray-dried material produced from each suspension was collected and placed into a glass vial and stored under dessication at room temperature.

3 Particle Size Analysis:

The particle size of both micronised BDP and spray dried BDP was determined in a liquid medium by laser diffraction, according to an independent model, using a Malvern 2600 laser diffraction sizer (Malvern Instruments, Malvern, Worcs, UK). BDP was measured using a 63 mm lens, after dispersion in a solution of 1% (w/v) span 85 in cyclohexane, saturated with the drug. Each sample was measured in triplicate.

4. Scanning Electron Microscopy of Spray-dried Particles

Double sided adhesive tape was placed on an aluminium stub and after stripping off the protective covering, a small amount of particles was scattered on the stub and dispersed by tapping lightly on the edge of the stub with a spatula to break up any agglomerates. The particles were then coated with approximately 15 to 20 nm gold using a sputter coater (Polaron E5100, Polaron Equipment Ltd, Watford, UK) with an electrical potential of 2.0 kV and a current of 20 mA. Several photomicrographs were produced by scanning fields, selected randomly, at different magnifications under a Philips SEM501B scanning electron microscope (Einhoven, Holland).

5. Preparation of Dry Powder Formulations:

Three different formulations were prepared. Two formulations used spray-dried material, the individual formulations containing different amounts of PVA, whilst the third employed micronised BDP alone. Each formulation was prepared by mixing the drug with sieved lactose as described below.

For each formulation sufficient powder mixture for 50 capsules was prepared. Initially a weight of the spray-dried powder was accurately weighed out into a tarred glass vial and sealed. The spray-dried powder was triturated in increasing quantities with sieved lactose (1.35 g) using a micro-spatula. After the powder had been blended in the vial, additional mixing was carried out in a Turbula® mixer for 30 min.

6. High Performance Liquid Chromatography (HPLC):

A validated HPLC method was employed to assay BDP. The assay conditions are presented in summary form below:

| | |
|---|---|
| Column: | 15 cm ODS Waters column |
| Mobile phase: | 70 methanol:30 water |
| Flow Rate: | 0.8 ml/min |
| Detector Wavelength: | UV at 239 nm |
| Loop Volume: | 50 μL |
| Pressure: | 975 p.s.i |
| Rheodyne Value | 7010 |
| Pump: | Constametric 3200 LDC Analytical |
| Detector: | Spectromonitor 3100 LDC Analytical |
| Integrator: | CI-400 LDC Analytical |
| Temperature: | ambient |
| Retention time: | approximately 4 min |
| Total run time: | 8 min |

7. In-vitro Deposition Using the Twin Stage Impinger Device (TSI):

The apparatus used conformed to the BP specifications and was washed thoroughly with distilled water and placed in a drying oven for 20 min before any deposition studies were carried out. Mobile phase (7 ml) was placed in the upper stage of the device and 30 ml of mobile phase was placed in the lower stage of the device. A moulded rubber adapter was attached to the mouth piece and the rest of the apparatus was aligned along the horizontal axis of the mouth piece. The outlet to the lower stage of the device was attached to an air pump which was calibrated to a flow rate of 60±5 L/min. Calibration was carried out using a Flowmeter.

The Rotahaler® device was attached to the adapter at the mouthpiece and a capsule was placed in the square orifice of the device. The lower half of the device was twisted to break open the capsule and release the dose of drug. Air was drawn through the device and impinger for 7 s. The pump was switched off and the lower half of the device was slowly separated from the rest of the device still attached to the adapter. Using forceps the gelatine capsule shell and any drug remaining in it were removed and placed in a beaker. This procedure was repeated for the remaining 2 capsules, so that for each deposition determination 3 capsules were employed.

After actuation of all three capsules the TSI was dismantled and washed with mobile phase and the washing solutions assayed separately.

The concentration of BDP was determined from calibration curves constructed using the standard solutions of BDP. It was possible to determine the amount of drug present in each section of the TSI and the amount associated with the device and capsule.

The following parameters were then calculated:

Recovered Dose (RD)=Drug determined in (Upper stage+Lower stage+Device+Capsule)

Emitted dose (ED)=Drug determined in (Upper stage+Lower stage)

Fine Particle Dose (FPD)=Drug determined in Lower stage

$$\begin{matrix}Fine Particle Fraction ( FPF ) = \frac{FPD}{RD} \\ \% Dispersibility = \frac{FPD}{ED} \\ \% Emission = \frac{ED}{RD} \\ \% Recovery = \frac{RD}{Original dose in capsule}\end{matrix}$$

### RESULTS

1) Particle Size

The volume mean diameters (VMD(GSD)) of the lactose crystals, micronised BDP and spray dried BDP were found to be 90.95(1.52) μm, 5.18 (1.00) μm and 6.43 (0.98) μm respectively.

2) Scanning Electron Microscopy

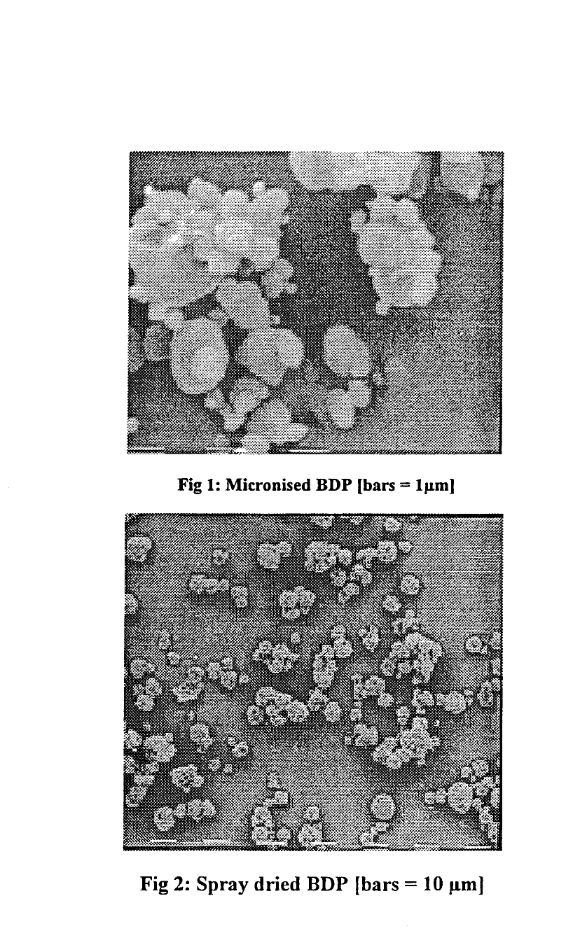

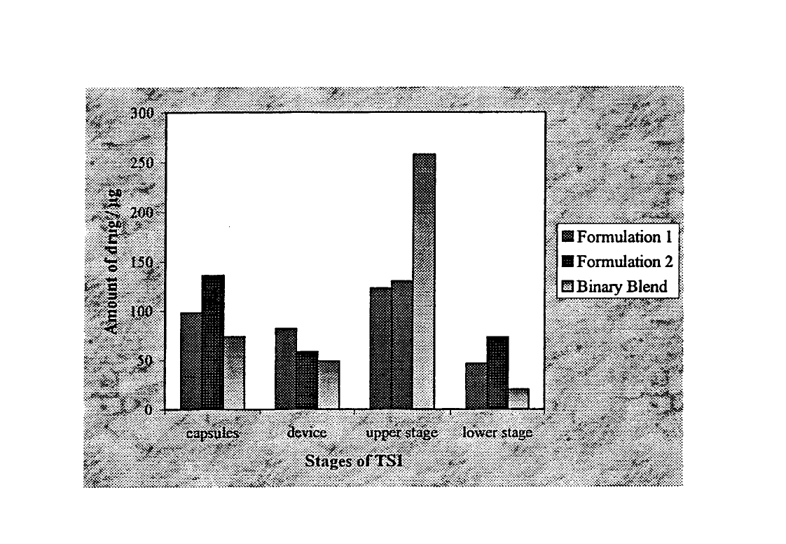

FIG. 1 shows micronised drug which, although smooth in appearance, tended to exist as agglomerated particles. In contrast the spray-dried BDP particles existed as individual spherical particles having a somewhat ‘spongy’ appearance (FIG. 2).

3) Deposition Profiles of BDP from Formulations 1, 2 and the Binary Blend of BDP with Coarse Lactose:

Powder formulations containing spray dried BDP (formulations 1 & 2) and the binary blend of BDP and coarse lactose (63-90 μm) were shown to produce different deposition profiles of BDP when aerosolised into a twin stage impinger (TSI) (Tables 1 to 3). The recovered dose (RD) was 349.5 μg for the formulation of BDP spray dried with 0.05% w/v PVA (Formulation 1), 397.1 μg for the formulation of BDP spray-dried with 0.1% PVA (Formulation 2) and 399.9 μg for the binary blend, corresponding to a % recovery of between 100% and 104%. The emitted dose (ED) of BDP ranged from 169.6 μg for Formulation 1 to 277.5 μg for binary blend, corresponding to an emission between 48.5% to 69.4%.

The results are shown in FIG. 3.

The binary blend of drug alone mixed with lactose produced the highest emission of the drug, however the resultant FPD and FPF was three to four times lower than that produced by the formulations containing the spray-dried drug (Tables 1 to 3). These results showed that a higher amount of the drug was deposited in the upper stage of the TSI from the control blend containing micronised drug than from the formulations containing modified drug.

TABLE 1
The recovered dose (RD), the emitted dose (ED), the fine particle dose (FPD), the fine particle
fraction (FPF) and the percentage dispersibility, emission and recovery of BDP from
Formulation 1, containing 0.073% w/w PVA

| | Dose of Drug/ | RD | ED | FPD | | % | % | % |
|---|---|---|---|---|---|---|---|---|
| Function | Capsule (μg) | (μg) | (μg) | (μg) | FPF | Dispersibility | Recovery | Emission |
| Mean | 336.24 | 349.54 | 169.59 | 46.69 | 13.35 | 27.4 | 103.96 | 48.51 |
| value | | | | | | | | |
| Stdev | 15.07 | 8.78 | 10.16 | 6.59 | 1.79 | 2.42 | 2.32 | 2.35 |

TABLE 2
The recovered dose (RD), the emitted dose (ED), the fine particle dose (FPD), the fine particle fraction
(FPF) and the percentage dispersibility, emission and recovery of BDP from Formulation 2, containing
0.146% w/w PVA:

| | Dose of Drug/ | RD | ED | FPD | | % | % | % |
|---|---|---|---|---|---|---|---|---|
| Function | Capsule (μg) | (μg) | (μg) | (μg) | FPF | Dispersibility | Recovery | Emission |
| Mean | 382.35 | 397.06 | 202.94 | 72.88 | 18.36 | 30.2 | 103.84 | 51.18 |
| value | | | | | | | | |
| St.dev | 6.14 | 9.13 | 28.79 | 9.001 | 2.28 | 9.98 | 2.38 | 7.78 |

TABLE 3
The recovered dose (RD), the emitted dose (ED) the fine particle dose (FPD) the fine particle fraction
(FPF) and the percentage dispersibility, emission and recovery of BDP from Binary Blend of drug
and lactose:

| | Dose of Drug/ | RD | ED | FPD | | % | % | % |
|---|---|---|---|---|---|---|---|---|
| Function | Capsule (μg) | (μg) | (μg) | (μg) | FPF | Dispersibility | Recovery | Emission |
| Mean | 399.09 | 399.88 | 277.50 | 20.20 | 5.05 | 7.29 | 100.19 | 69.44 |
| value | | | | | | | | |
| Stdev | 9.08 | 12.46 | 12.22 | 1.43 | 0.36 | 0.67 | 3.12 | 3.87 |

### DISCUSSION

The results from this study demonstrate that it is possible to transform a micronised hydrophobic drug, the particles of which aggregate together in the dry state, to a powder that is comprised primarily of individual particles. This was achieved by spray-drying the drug with a low concentration of PVA, an excipient currently employed in pressurised multidose inhaler formulations. The total amounts of PVA employed in this study comprised either 0.073% or 0.146% of the final powder mass, thus the dose of PVA in each capsule was either 20 or 40 μg, depending upon the polymer concentration employed in the spray-drying procedure. After spray-drying it was possible to blend the drug with lactose to obtain a uniform powder mixture. The aerosolisation of the powders at 60 L min⁻¹ from a Rotahaler into a TSI resulted in the FPD of BDP being increased from 20.2 μg when micronised drug was employed to 46.7 μg when the lower PVA concentration was employed. The FPD was increased further, to 72.9 μg, when the formulation containing the drug spray-dried with the higher concentration of PVA was aerosolised.

The results obtained in this study suggest that the approach of spray-drying a hydrophobic drug with small quantities of a pharmaceutically acceptable hydrophilic polymer holds great promise in the formulation of other, similar materials as dry powders intended for pulmonary delivery. Conversely the spray drying of a hydrophilic drug with a hydrophobic polymer might provide a means of providing protection against moisture uptake and improve flow and handling properties. Suitable hydrophobic polymers include modified celluloses (such as ethylcellulose) and acrylic polymers (such as methyl methacrylate). An alternative strategy might be to spray dry such a drug from solution including a surfactant with a low HLB, such as a Span.

### REFERENCES

1. British Asthma Guidelines Coordination Committee. 1997. British Asthma Guidelines on Asthma Management. Thorax. 52 (Suppl. 1): S1-S21.

2. Higgins, G. 1997. New US asthma guidelines stress early, aggressive treatment. Inpharma. 1077: 9-10.

3. Keeley, D. and J. Rees. 1997. New Guidelines on asthma management: aim to control symptoms rapidly, with higher initial doses of steroid and earlier use of beta agonists. Br. Med J. 314:315-316.

4. Dluhy, R. G. 1998. Effect of inhaled beclomethasone dipropionate and budesonide on adrenal function, skin changes and cataract formation. Respiratory Medicine. 92(Suppl. B): 15-23.

5. Timsina, M. P., G. P. Martin, C. Marriott, D. Ganderton and M. Yianneskis. 1994. Drug delivery to the respiratory tract using dry powder inhalers. Int. J. Pharm. 101: 1-13.

6. Pauwels, R., S. Newman, and L. Borgstrom. 1997. Airway deposition and airway effects of antiasthma drugs delivered from metered-dose inhalers. Eur. Respir. J. 10: 2127-2138.

7. Gupta, P. K., and A. J. Hickey. 1991. Comtemporary approaches in aerosolized drug delivery to the lung. J. Controlled Rel. 17: 129-148.

8. Kassem, N. M. 1990. Generation of deeply inspirable dry powders, PhD thesis, University of London.

1. A particulate pharmaceutical composition for pulmonary delivery, which comprises a hydrophobic pharmaceutically active agent which has been spray-dried with a small quantity of a pharmaceutically acceptable hydrophilic polymer to form spray-dried particles.

2. A composition according to claim 1 wherein the ratio of active agent to hydrophilic polymer is in the range 1:0.05 to 1:0.1 in the spray-dried particles.

3. A composition according to any preceding claim wherein the hydrophilic polymer is poly(vinyl)alcohol.

4. A composition according to claim 3 wherein the poly(vinyl)alcohol has a molecular weight of 9000 to 10,000.

5. A composition according to any preceding claim wherein the volume mean diameter of the spray-dried particles is from 1 to 6.4 μm.

6. A composition according to any preceding claim wherein the pharmaceutically active agent is a corticosteroid.

7. A composition according to claim 6 wherein the corticosteroid is beclomethasone dipropionate.

8. A composition according to claim 6 wherein the corticosteroid is budesonide or fluticosone dipropionate.

9. A composition according to any of claims 1 to 5 wherein the pharmaceutically active agent is a beta 2 agonist.

10. A composition according to claim 9 wherein the beta 2 agonist is salmeterol or salbutamol.

11. A composition according to any of claims 1 to 5 wherein the pharmaceutically active agent is an anticholinergic drug.

12. A composition according to claim 11 wherein the anticholinergic drug is ipratropium bromide.

13. A composition according to any of claims 1 to 5 wherein the pharmaceutically active agent is a leukotriene.

14. A composition according to any of claims 1 to 5 wherein the pharmaceutically active agent is a peptide, an antisense therapeutic or a gene therapeutic.

15. A composition according to claim 14 wherein the peptide is insulin or calcitonin.

16. A composition according to any preceding claim, which further comprises a pharmaceutically acceptable carrier.

17. A composition according to claim 16 wherein the carrier is lactose.

18. A composition according to claim 16 or 17 wherein the carrier has a specific size range of 63-90 μm.

19. A composition according to any of claims 16 to 18 wherein the ratio of spray-dried particles to carrier is substantially 1:67.5 w/w.

20. A composition according to any of claims 16 to 19 wherein the hydrophilic polymer is 0.073 to 0.146% of the total composition weight.

21. A method of producing a particulate pharmaceutical composition, which comprises spray drying a hydrophobic pharmaceutically active agent with a small quantity of a pharmaceutically acceptable hydrophilic polymer to form spray-dried particles.

22. A method according to claim 21 wherein active agent particles are dispersed in a solution of the hydrophilic polymer, prior to spray drying.

23. A particulate pharmaceutical composition for pulmonary delivery, which comprises a hydrophobic pharmaceutically active agent which has been spray-dried with a small quantity of a pharmaceutically acceptable hydrophilic polymer; or conversely a hydrophilic pharmaceutically active agent which has been spray-dried with a small quantity of a pharmaceutically acceptable hydrophobic polymer; to form spray-dried particles.
